(19) United States
(12) Patent Application Publication
(10) Pub. No.: US 2026/0021241 A1
(43) Pub. Date: Jan. 22, 2026

(54) PORTABLE HAND-HELD NEGATIVE PRESSURE NASAL IRRIGATOR

(71) Applicant: Nicefeel Medical Device Technology Co.,Ltd., Shenzhen (CN)

(72) Inventor: Xiao LIU, Shenzhen (CN)

(21) Appl. No.: 19/083,101

(22) Filed: Mar. 18, 2025

(30) Foreign Application Priority Data
Jul. 17, 2024 (CN) 202410963861.3

(51) Int. Cl. A61M 3/02

(52) U.S. Cl. CPC A61M 3/0245; A61M 3/0202; A61M 3/0258; A61M 2205/3337; A61M 2205/75; A61M 2205/8206; A61M 2210/0618

(57) ABSTRACT

A portable hand-held negative pressure nasal irrigator comprises a clean water tank, a machine body, left and right nasal interfaces, a vacuum pump, an intermediate adapter seat and a waste water tank. The left and right nasal interfaces are inserted into the machine body, the clean water tank is located in the machine body, the intermediate adapter seat is located below the machine body, the waste water tank is located in the intermediate adapter seat, a left nasal interface of the left and right nasal interfaces is communicated with the clean water tank, a right nasal interface of the left and right nasal interfaces is communicated with the waste water tank, the clean water tank and the waste water tank are connected by the vacuum pump, and an air suction end of the vacuum pump is at a top portion of the waste water tank.

### CROSS REFERENCE TO RELATED APPLICATIONS

This application claims foreign priority of Chinese Patent Application No. 202410963861.3, filed on Jul. 17, 2024 in the China National Intellectual Property Administration, the disclosures of all of which are hereby incorporated by reference.

### TECHNICAL FIELD

The present invention relates to the technical field of medical instruments, and particularly to a portable hand-held negative pressure nasal irrigator.

### BACKGROUND OF THE PRESENT INVENTION

Due to the pollution of ambient air and the frequent occurrence of Covid-19 and respiratory diseases in recent years, people pay more and more attention to nasal respiratory tract care, and the nasal washing method in daily life can no longer meet people's requirements for nasal care. The principle of nasal irrigator is nasal irrigation with saline water, and a lot of allergens, bacteria, viruses and pus accumulated in a nasal cavity, especially in a paranasal sinus, are eliminated through the anti-inflammatory and bactericidal effects of saline water to give the nasal cavity a clean and refreshing environment, thus restoring the function of nasal cilia and accelerating the recovery of health. For general allergic rhinitis, frequent nasal irrigation can increase the tolerance of nasal cavity to allergens and gradually make the nasal cavity lose its allergic reaction to the allergens.

As a tool for cleaning the nasal cavity, the nasal irrigator usually delivers normal saline into a nostril through a certain pressure (or suction such as a Dianbang Airbag nasal irrigator, or gravity such as a simple bottle or jar nasal irrigator, or a mechanical pressure such as an electric nasal irrigator), and the normal saline flows through a nasal vestibule (a part exposed outside the head), a paranasal sinus and a nasal passage, bypasses a nasopharynx, and is discharged from one nostril or mouth. However, at present, most nasal irrigators are complicated in structure, bulky and inconvenient to carry, and waste water easily enters a vacuum pump, which affects the service life, resulting in poor user experience of common nasal irrigators. Therefore, we invented a new portable hand-held negative pressure nasal irrigator, which solved the above technical problems.

### SUMMARY OF THE PRESENT INVENTION

The present invention aims to provide a portable hand-held negative pressure nasal irrigator which is simple and light, portable, low in cost and convenient to use.

The objective of the present invention can be achieved by the following technical means: a portable hand-held negative pressure nasal irrigator is provided, wherein the portable hand-held negative pressure nasal irrigator comprises a clean water tank, a machine body, left and right nasal interfaces, a vacuum pump, an intermediate adapter seat and a waste water tank, the left and right nasal interfaces are inserted into the machine body, the clean water tank is located in the machine body, the intermediate adapter seat is located below the machine body, the waste water tank is located in the intermediate adapter seat, a left nasal interface of the left and right nasal interfaces is communicated with the clean water tank, a right nasal interface of the left and right nasal interfaces is communicated with the waste water tank through a pipeline inside the machine body, the clean water tank and the waste water tank are connected by the vacuum pump, an air suction end of the vacuum pump is at a top portion of the waste water tank, and an air outlet of the vacuum pump is at a top portion of the clean water tank.

The objective of the present invention can also be achieved by the following technical means:
the portable hand-held negative pressure nasal irrigator further comprises an automatic one-way valve, and the automatic one-way valve is located in the left nasal interface, so as to prevent water from flowing out of the left nasal interface due to gravity after the clean water tank is full of water.

The portable hand-held negative pressure nasal irrigator further comprises an air outlet one-way valve, the air outlet one-way valve is located at an air outlet in the clean water tank and connected with the air outlet of the vacuum pump, so as to prevent water in the clean water tank from entering the vacuum pump in the case of turning off when the clean water tank is full of water.

The portable hand-held negative pressure nasal irrigator further comprises a self-made one-way valve, the self-made one-way valve is located in the intermediate adapter seat, and after the waste water tank is full of water, the self-made one-way valve blocks an air suction port of the vacuum pump with the buoyancy of water and the suction of the vacuum pump, so as to prevent waste water from being sucked into the vacuum pump.

The portable hand-held negative pressure nasal irrigator further comprises a small water tank cover, a filter screen and a large water tank cover, after the filter screen is mounted below the small water tank cover, the small water tank cover is mounted on the large water tank cover to filter a liquid entering the clean water tank, and the large water tank cover is located above the clean water tank to seal the clean water tank.

The machine body and the left and right nasal interfaces are in a cylindrical inserting structure, so that the left and right nasal interfaces are capable of rotating by 360 degrees.

The portable hand-held negative pressure nasal irrigator further comprises a left and right nasal interface sleeve, and the left and right nasal interface sleeve is sleeved on the left and right nasal interfaces, so as to maintain the hygiene of the left and right nasal interfaces.

The clean water tank is located in the machine body and integrally designed with the machine body, and a bottom portion of the machine body is ultrasonically connected with the intermediate adapter seat and hermetically connected with the waste water tank in a clamped way.

The portable hand-held negative pressure nasal irrigator further comprises a switch button, a battery compartment and a lithium battery, the switch button is connected to the battery compartment, the lithium battery is mounted in the battery compartment and the battery compartment is connected to the vacuum pump, and when the switch button is pressed, the lithium battery supplies power to the vacuum pump, so that the vacuum pump starts to work.

The portable hand-held negative pressure nasal irrigator further comprises a machine core assembly, the machine core assembly is connected with the battery compartment and the switch button, and the machine core assembly controls a rotating speed and a gear of the vacuum pump under the control of the switch button.

The portable hand-held negative pressure nasal irrigator further comprises a USB charging seat, a USB charging cable and a USB seat cover, the USB charging seat is connected to the battery compartment, and an external power supply is connected to the USB charging seat through the USB charging cable, so as to charge the lithium battery in the battery compartment; and the USB seat cover covers the USB charging seat, so as to realize waterproof and dustproof.

According to the portable hand-held negative pressure nasal irrigator in the present invention, the self-made valve is designed at the air suction port of the vacuum pump to prevent water from being sucked into the vacuum pump, so that the service life of the nasal irrigator is prolonged. The one-way valve is arranged in a pipe of the air outlet of the vacuum pump, so that water will not flow out under natural gravity of water after the clean water tank is full of water. According to the portable hand-held negative pressure nasal irrigator, air suction and discharge of the vacuum pump are combined into one “pipeline” through the vacuum pump, and clean water may be pumped out to the waste water tank by the pipeline through one nasal cavity to the other nasal cavity of human body, so that the portable hand-held negative pressure nasal irrigator has the advantages of simple internal structure, simple manufacturing process, small volume, operation by a user with one hand, portability, charging at any time, low cost and beautiful appearance.

### DESCRIPTION OF THE DRAWINGS

FIG. 1 is a schematic structural diagram of one specific embodiment of a portable hand-held negative pressure nasal irrigator of the present invention;

FIG. 2 is a rear view of the portable hand-held negative pressure nasal irrigator in one specific embodiment of the present invention;

FIG. 3 is a working diagram of a self-made valve of the portable hand-held negative pressure nasal irrigator in one specific embodiment of the present invention; and

FIG. 4 is a working diagram of an automatic one-way valve of the portable hand-held negative pressure nasal irrigator in one specific embodiment of the present invention.

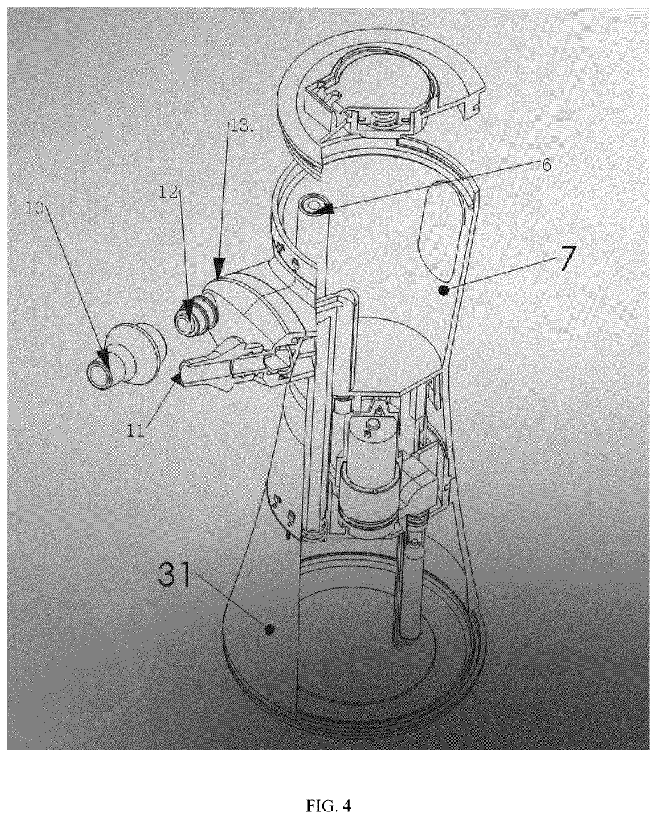

### DETAILED DESCRIPTION OF PREFERRED EMBODIMENTS

It should be noted that the following detailed descriptions are all exemplary and are intended to further describe the present invention. Unless otherwise specified, all technical and scientific terms used herein have the same meanings as those commonly understood by those of ordinary skills in the art to which the present invention belongs.

It should be noted that the terms used herein are only used to describe specific embodiments, and are not intended to limit exemplary embodiment according to the present invention. As used herein, the singular form is intended to include the plural form, unless the context clearly indicates otherwise. In addition, it should be further understood that terms “include” and/or “comprise” used in this specification indicate that there are features, steps, operations, and/or combinations thereof.

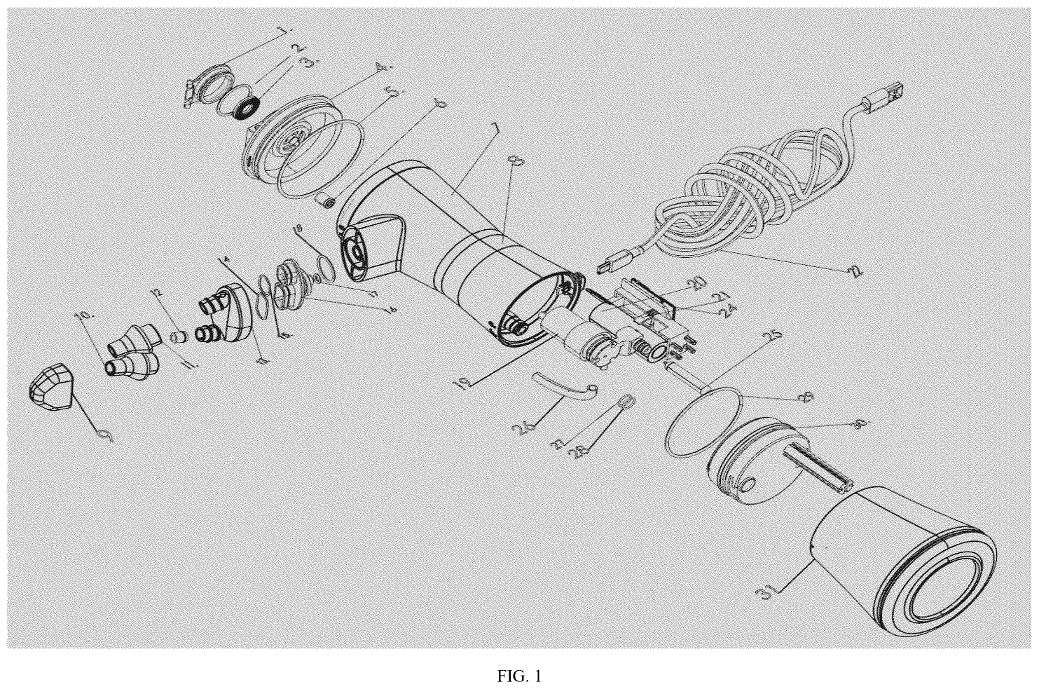

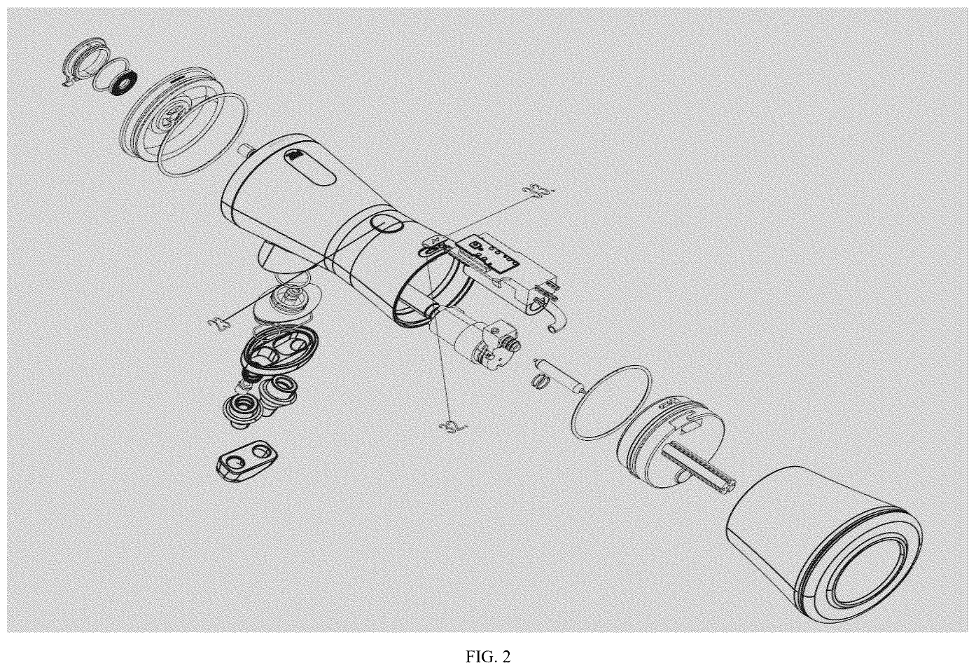

FIG. 1 and FIG. 2 show is a structural diagram of a portable hand-held negative pressure nasal irrigator of the present invention. The portable hand-held negative pressure nasal irrigator comprises a small water tank cover 1, a sealing ring 2, a filter screen 3, a large water tank cover 4, a sealing ring 5, an air outlet one-way valve 6, a clean water tank 7, a machine body 8, a left and right nasal interface sleeve 9, a left nasal interface 10, a right nasal interface 11, an automatic one-way valve 12, a left and right nasal sleeve cover 13, a left nasal inner pipe sealing ring 14, a right nasal inner pipe sealing ring 15, a left and right nasal sleeve panel assembly 16, a sealing ring 17, a sealing ring 18, a vacuum pump 19, a lithium battery 20, a battery compartment 21, a USB charging cable 22, a switch button 23, a machine core assembly 24, a self-made air inlet valve 25, a rubber pipe 26, a sealing ring 27, a sealing ring 28, a sealing ring 29, an intermediate adapter seat 30, a waste water tank 31, a USB seat cover 32, and a USB charging seat 33.

After the filter screen 3 is mounted below the small water tank cover 1 through the sealing ring 2, the small water tank cover is mounted on the large water tank cover 4 to filter a liquid entering the clean water tank 7. The large water tank cover 4 is located above the clean water tank 7 to seal the clean water tank 7 through the sealing ring 5. The clean water tank 7 is located in the machine body 8, and is designed integrally with the machine body 8. Front ends of the left and right nasal interfaces are the left nasal interface 10 and the left nasal interface 11 which may be inserted into left and right nasal cavities, and rear ends of the left and right nasal interfaces are connected to the clean water tank 7. Waterproof silicone rings are arranged on the left and right nasal interfaces, wherein the waterproof silicone ring 17 is arranged on the left nasal interface, and the waterproof silicone ring 18 is arranged on the right nasal interface, and the left and right nasal interfaces are directly inserted into the machine body. The machine body and the left and right nasal interfaces are in a cylindrical inserting structure, so that the left and right nasal interfaces are capable of rotating by 360 degrees at the same time. Specifically, the automatic one-way valve 12, the left and right nasal sleeve cover 13, the left nasal inner pipe sealing ring 14, the right nasal inner pipe sealing ring 15, the left and right nasal sleeve panel assembly 16, the sealing ring 17 and the sealing ring 18 are all rotatable, and are “integrated”.

The left nasal interface 10 and the right nasal interface 11 are made of a rubber material, and sleeved on the left and right nasal sleeve cover 13, the left and right nasal sleeve cover 13 is connected to the left and right nasal sleeve panel assembly 16 through the left nasal inner pipe sealing ring 14 and the right nasal inner pipe sealing ring 15, and the left and right nasal sleeve panel assembly 16 is inserted into the machine body 8 through the sealing ring 17 and the sealing ring 18.

The left and right nasal interface sleeve 9 is sleeved on the left nasal interface 10 and the right nasal interface 11 to keep the interfaces clean and hygienic when not in use.

The automatic one-way valve 12 is located in the left and right nasal sleeve cover 13 and the left nasal interface 10, so as to prevent water from flowing out of the left nasal interface 10 due to gravity after the clean water tank 7 is full of water.

The air outlet one-way valve 6 is located at an air outlet in the clean water tank 7 and connected with the air outlet of the vacuum pump 19, so as to prevent water in the clean water tank 7 from entering the vacuum pump 19 in the case of turning off when the clean water tank 7 is full of water.

The intermediate adapter seat 30 is located below the machine body 8, the waste water tank 31 is located in the intermediate adapter seat 30, and a bottom portion of the machine body 8 is ultrasonically connected with the intermediate adapter seat 30 and hermetically connected with the waste water tank 31 in a clamped way.

One end of the rubber pipe 26 is connected to the air outlet of the vacuum pump, and the other end of the rubber pipe is connected to the clean water tank 7 through the sealing ring 27 and the sealing ring 28.

The intermediate adapter seat 30 is connected to that bottom portion of the machine body 8 through the sealing ring 29.

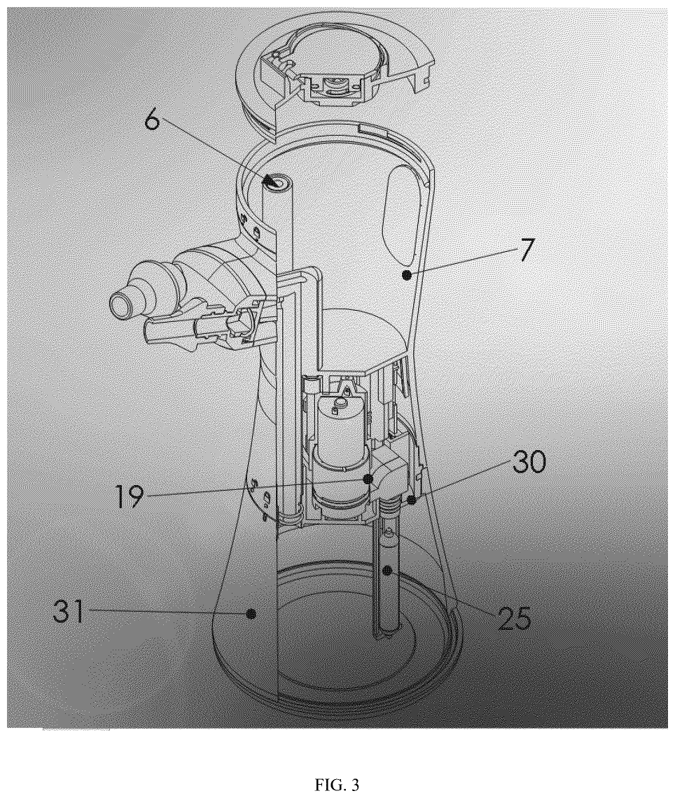

As shown in FIG. 3, the self-made air inlet valve 25 is located in a funnel of the intermediate adapter seat 30. After the waste water tank 31 is full of water, the self-made one-way valve 25 will block an air suction port of the vacuum pump 19 with the buoyancy of water and the suction of the vacuum pump 19, so as to prevent waste water from entering the vacuum pump 19.

The lithium battery 20 is located in the battery compartment 21, the switch button 23 is connected to the battery compartment 21, the battery compartment 21 is connected to the vacuum pump 19, and when the switch button 23 is pressed, the lithium battery 20 supplies power to the vacuum pump 19, so that the vacuum pump 19 starts to work.

The machine core assembly 24 is connected to the battery compartment 21 and the switch button 23, and the machine core assembly controls a rotating speed and a gear of the vacuum pump under the control of the switch button 23. The switch button 23 is pressed for the first time to turn on, pressed for the second time to switch to a first gear, pressed for the third time to switch to a second gear, pressed for the fourth time to switch to a third gear, and pressed for the fifth time to turn off. These switching functions are programmed by the machine core assembly 24.

The USB charging seat 33 is connected to the battery compartment 21, and an external power supply is connected to the USB charging seat 33 through the USB charging cable 22 to charge the lithium battery 20 in the battery compartment 21.

The USB seat cover 32 covers the USB charging seat 33 to realize dustproof.

When the portable hand-held negative pressure nasal irrigator of the present invention is used, the left nasal interface is communicated with the clean water tank, and the right nasal interface is communicated with the waste water tank below through a pipeline inside the machine body. The clean water tank and the waste water tank are connected by the vacuum pump, an air suction end of the vacuum pump is at a top portion of the waste water tank, and the air outlet of the vacuum pump is at a top portion of the clean water tank. When in use, the vacuum pump starts up, there is a positive pressure in the clean water tank, and there is a negative pressure in the waste water tank, so that one sealed “pipeline” is formed with nasal cavities of human body.

A lower end of the vacuum pump is connected to an air inlet, and the air inlet is provided with the self-made one-way valve and located in the waste water tank. When the vacuum pump starts to work, there is the positive pressure in the clean water tank, and there is the negative pressure in the waste water tank. Under an action of the positive pressure, water in the clean water tank enters the left nasal interface, and then enters the nasal cavities through the left nasal sleeve inserted into the left nasal cavity. The water after cleaning the nasal cavities enters the right nasal interface through the right nasal sleeve inserted into the right nasal cavity, and then enters the waste water tank through the pipeline inside the machine body.

After the waste water tank is full of water, the self-made one-way valve at the air inlet blocks the air suction port of the vacuum pump with the buoyancy of water and the suction of the vacuum pump, so as to prevent water from entering the vacuum pump. When there is no water in the waste water tank and there is no suction, the self-made one-way valve at the air inlet falls with its own gravity to a lower end of the funnel of the intermediate adapter seat.

When assembling, the self-made one-way valve is mounted in the funnel of the intermediate adapter seat first. After the waste water tank is full of water, the self-made one-way valve will block the air suction port of the vacuum pump with the buoyancy of water and the suction of the vacuum pump, so as to prevent waste water from entering the vacuum pump.

The automatic one-way valve is designed at a water outlet in the left and right nasal sleeve cover, so that water will not flow out of the left nasal interface due to gravity after the water tank is full of water. For the water in the clean water tank, only when the vacuum pump is turned on, the air suction port of the vacuum pump pumps air from the waste water tank, and the air flows out of the air outlet to form a pressure in the clean water tank. At this time, the automatic one-way valve is pushed open.

Compared with the prior art, the present invention has the following technical features.

1. The self-made valve is designed at the air suction port of the waste water tank, so as to prevent waste water from entering the vacuum pump after the waste water tank is full of water.

2. The one-way valve is designed at the air outlet of the clean water tank, so as to prevent clean water from being added before turning off to enter the vacuum pump after the clean water tank is full of water.

3. The automatic one-way valve is designed at a left water outlet of the left and right nasal sleeve cover, so as to prevent clean water from being added before turning on to flow out of the left water outlet with gravity after the clean water tank is full of water.

Finally, it should be noted that: the above are only the preferred embodiments of the present invention, and the preferred embodiments are not intended to limit the present invention. Although the present invention is described in detail with reference to the above embodiments, those skilled in the art may still modify the technical solutions recorded in the above embodiments, or make equivalent replacements to some of the technical features. Any modification, equivalent substitution, improvement, etc. made within the spirit and principle of the present invention are included in the scope of protection of the present invention.

Except for the technical features described in the specification, they are all known to those skilled in the art.

We claim:

1. A portable hand-held negative pressure nasal irrigator, wherein the portable hand-held negative pressure nasal irrigator comprises a clean water tank, a machine body, left and right nasal interfaces, a vacuum pump, an intermediate adapter seat and a waste water tank, the left and right nasal interfaces are inserted into the machine body, the clean water tank is located in the machine body, the intermediate adapter seat is located below the machine body, the waste water tank is located in the intermediate adapter seat, a left nasal interface of the left and right nasal interfaces is communicated with the clean water tank, a right nasal interface of the left and right nasal interfaces is communicated with the waste water tank through a pipeline inside the machine body, the clean water tank and the waste water tank are connected by the vacuum pump, an air suction end of the vacuum pump is at a top portion of the waste water tank, and an air outlet of the vacuum pump is at a top portion of the clean water tank.

2. The portable hand-held negative pressure nasal irrigator according to claim 1, wherein the portable hand-held negative pressure nasal irrigator further comprises an automatic one-way valve, and the automatic one-way valve is located in the left nasal interface, so as to prevent water from flowing out of the left nasal interface due to gravity after the clean water tank is full of water.

3. The portable hand-held negative pressure nasal irrigator according to claim 1, wherein the portable hand-held negative pressure nasal irrigator further comprises an air outlet one-way valve, the air outlet one-way valve is located at an air outlet in the clean water tank and connected with the air outlet of the vacuum pump, so as to prevent water in the clean water tank from entering the vacuum pump in the case of turning off when the clean water tank is full of water.

4. The portable hand-held negative pressure nasal irrigator according to claim 1, wherein the portable hand-held negative pressure nasal irrigator further comprises a self-made one-way valve, the self-made one-way valve is located in the intermediate adapter seat, and after the waste water tank is full of water, the self-made one-way valve blocks an air suction port of the vacuum pump with the buoyancy of water and the suction of the vacuum pump, so as to prevent waste water from being sucked into the vacuum pump.

5. The portable hand-held negative pressure nasal irrigator according to claim 1, wherein the portable hand-held negative pressure nasal irrigator further comprises a small water tank cover, a filter screen and a large water tank cover, after the filter screen is mounted below the small water tank cover, the small water tank cover is mounted on the large water tank cover to filter a liquid entering the clean water tank, and the large water tank cover is located above the clean water tank to seal the clean water tank.

6. The portable hand-held negative pressure nasal irrigator according to claim 1, wherein the machine body and the left and right nasal interfaces are in a cylindrical inserting structure, so that the left and right nasal interfaces are capable of rotating by 360 degrees.

7. The portable hand-held negative pressure nasal irrigator according to claim 1, wherein the portable hand-held negative pressure nasal irrigator further comprises a left and right nasal interface sleeve, and the left and right nasal interface sleeve is sleeved on the left and right nasal interfaces, so as to maintain the hygiene of the left and right nasal interfaces.

8. The portable hand-held negative pressure nasal irrigator according to claim 1, wherein the clean water tank is located in the machine body and integrally designed with the machine body, and a bottom portion of the machine body is ultrasonically connected with the intermediate adapter seat and hermetically connected with the waste water tank in a clamped way.

9. The portable hand-held negative pressure nasal irrigator according to claim 1, wherein the portable hand-held negative pressure nasal irrigator further comprises a switch button, a battery compartment and a lithium battery, the switch button is connected to the battery compartment, the lithium battery is mounted in the battery compartment and the battery compartment is connected to the vacuum pump, and when the switch button is pressed, the lithium battery supplies power to the vacuum pump, so that the vacuum pump starts to work.

10. The portable hand-held negative pressure nasal irrigator according to claim 9, wherein the portable hand-held negative pressure nasal irrigator further comprises a machine core assembly, the machine core assembly is connected to the battery compartment and the switch button, and the machine core assembly controls a rotating speed and a gear of the vacuum pump under the control of the switch button.

11. The portable hand-held negative pressure nasal irrigator according to claim 9, wherein the portable hand-held negative pressure nasal irrigator further comprises a USB charging seat, a USB charging cable and a USB seat cover, the USB charging seat is connected to the battery compartment, and an external power supply is connected to the USB charging seat through the USB charging cable, so as to charge the lithium battery in the battery compartment; and the USB seat cover covers the USB charging seat, so as to realize waterproof and dustproof.
